(19) United States
(12) Patent Application Publication
(10) Pub. No.: US 2022/0126063 A1
(43) Pub. Date: Apr. 28, 2022

(54) MEDICAL DEVICE WITH PRINTED BATTERY

(71) Applicant: BOSTON SCIENTIFIC SCIMED, INC., MAPLE GROVE, MN (US)

(72) Inventor: JAN WEBER, MAASTRICHT (NL)

(73) Assignee: BOSTON SCIENTIFIC SCIMED, INC., MAPLE GROVE, MN (US)

(21) Appl. No.: 17/512,277

(22) Filed: Oct. 27, 2021

Related U.S. Application Data
(60) Provisional application No. 63/106,138, filed on Oct. 27, 2020.

(51) Int. Cl. A61M 25/00

(52) U.S. Cl. CPC A61M 25/0097; A61M 25/0009; A61M 25/002; A61M 2205/8237; A61M 2205/8206; A61M 2205/3327; A61M 2205/3538; A61M 2025/0098

(57) ABSTRACT

A medical device such as a catheter includes a shaft that extends from a proximal region to a distal region and that defines a shaft lumen extending therethrough. A hub is secured relative to the proximal region. A strain relief extends proximally over the shaft from the hub. At least one of the hub and the strain relief include a printed battery.

### CROSS REFERENCE TO RELATED APPLICATIONS

The present application claims the benefit of and priority to U.S. Prov. Pat. App. No. 63/106,138, filed Oct. 27, 2020 and titled MEDICAL DEVICE WITH PRINTED BATTERY, the disclosure of which is incorporated herein by reference.

### TECHNICAL FIELD

The present disclosure pertains to medical devices, and methods for manufacturing and using medical devices. More particularly, the disclosure is directed to medical devices, and methods for manufacturing and using said medical devices, that include a printed battery forming part of the medical device.

### BACKGROUND

A number of medical devices include sensors, microprocessors, and other elements that require electrical power to function. While medical devices may include onboard batteries to supply electrical power, it will be appreciated that batteries sometimes tend to lose power during storage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. As an example, a catheter includes a shaft extending from a proximal region to a distal region and defining a shaft lumen extending therethrough. A hub is secured relative to the proximal region and a strain relief extends proximally over the shaft from the hub. At least one of the hub and the strain relief comprises a printed battery.

Alternatively or additionally, the hub may include the printed battery.

Alternatively or additionally, the hub may include hub wings that form a portion of the printed battery.

Alternatively or additionally, the hub includes a Luer fitting that provides an electrical connection to the printed battery.

Alternatively or additionally, the Luer fitting also provides a fluid coupling to the shaft lumen.

Alternatively or additionally, the catheter may further include a device coupled to the catheter that is powered by the printed battery.

Alternatively or additionally, the device may include one or more of a sensor, an actuator and an antenna.

Alternatively or additionally, the catheter may further include printed conductive traces that extend from the printed battery to the device in order to power the device.

Alternatively or additionally, the strain relief may include the printed battery.

Alternatively or additionally, the printed battery may include a printed current collector layer, a printed cathode layer, a printed electrolyte layer and a printed anode layer.

Alternatively or additionally, the printed battery may further include an encapsulating layer that forms at least a portion of an outer profile of the at least the hub or the strain relief.

As another example, a catheter system includes a catheter and a package that is adapted to secure the catheter within the package. The catheter includes a shaft extending from a proximal region to a distal region and defining a shaft lumen extending therethrough, a hub secured relative to the proximal region and a strain relief extending proximally over the shaft from the hub. The hub includes a printed catheter battery. The package includes a cavity adapted to accommodate the catheter and a printed package battery disposed within the package, the printed package battery electrically coupled with the printed catheter package in order to maintain an electrical charge within the printed catheter battery.

Alternatively or additionally, the catheter may include a Luer fitting that is electrically coupled with the printed catheter battery, and the printed package battery is electrically coupled with the printed catheter battery via the Luer fitting.

Alternatively or additionally, the Luer fitting may also provide a fluid coupling with the shaft lumen.

Alternatively or additionally, the catheter may further include a device coupled to the catheter that is powered by the printed catheter battery.

Alternatively or additionally, the device may include one or more of a sensor, an actuator and an antenna.

Alternatively or additionally, the catheter may further include printed conductive traces that extend from the printed catheter battery to the device in order to power the device.

As another example, a method of manufacturing a catheter includes providing a catheter shaft defining a shaft lumen, the catheter shaft including a proximal region and a distal region. A solid state polymer battery is formed over the proximal region of the catheter shaft, the solid state polymer battery formed as at least one of a hub for the catheter and a strain relief for the catheter. An electrically powered device is secured to the distal region of the catheter shaft. Electrical connectors are formed along the catheter shaft that electrically couple the device with the solid state polymer battery.

Alternatively or additionally, forming the solid state polymer battery may include printing a current collector layer, printing a cathode layer, printing an electrolyte layer and printing an anode layer.

Alternatively or additionally, the method may further include forming an encapsulating layer over the printed layers that forms at least a portion of an outer profile of the at least the hub or the strain relief.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an illustrative catheter;

FIG. 1A is a cross-sectional view of the illustrative catheter, taken along line 1A-1A of FIG. 1;

FIG. 2 is a schematic view of an illustrative catheter;

FIG. 3 is a schematic view of an illustrative catheter system;

FIG. 4 is a schematic view of an illustrative printed battery; and

FIG. 5 is a flow diagram showing an illustrative method of forming a catheter.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

A number of medical devices such as, but not limited to, catheters can include one or more electronic devices such as sensors, actuators, antennas, receivers and transmitters and the like. As a result, there is interest in being able to unobtrusively include a power supply as part of the medical device that can be used to power these electronic devices. Being able to incorporate the power supply into the medical device can mean that these electronic devices do not have to be connected to external power supplies. The cables needed to connect to external power supplies can be unwieldy and can potentially become entangled with other medical devices being used with a particular patient. If the power supply can be incorporated into the medical device in such a way as to not materially make the medical device bulkier, that is even better.

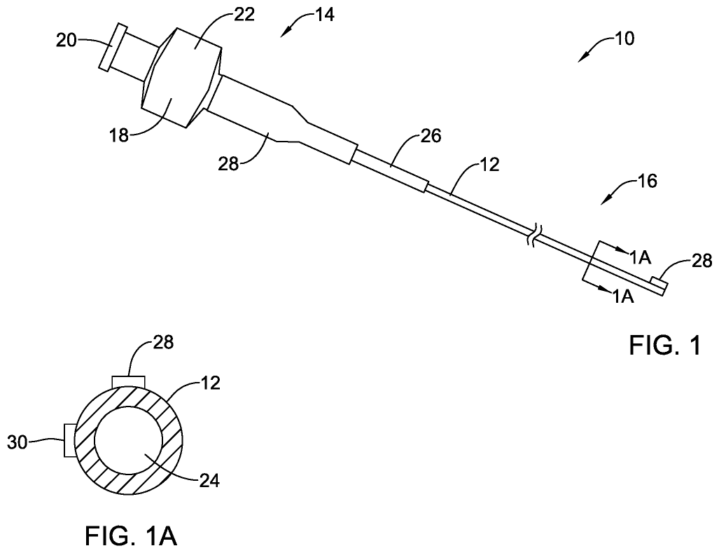

In some cases, a printed battery can be formed that replaces one or more components of the medical device. While the invention contemplates usage with any number of different medical devices, it will be appreciated that merely for illustrative purposes, the incorporation of a printed battery into a medical device is shown and described herein with respect to a catheter. FIG. 1 is a perspective view of an illustrative catheter 10. The illustrative catheter 10 includes a catheter shaft 12 that extends from a proximal region 14 to a distal region 16. The catheter 10 includes a hub 18 that is secured to the proximal region 14 of the catheter shaft 12. In some cases, as illustrated, the hub 18 includes a Luer fitting 20 and hub wings 22. The Luer fitting 20 provides fluid access to a shaft lumen 24, as shown in FIG. 1A.

The proximal region 14 of the catheter shaft 12 also includes a strain relief 26. In some cases, a tube cover 28 may extend over a portion of the hub 18 and the strain relief 26, but this is not required in all cases. The distal region 16 of the catheter shaft 12 includes a device 28 that is shown schematically as being operably coupled to the catheter 10 and that draws electrical power for operation. The device 28 can be a sensor, for example, or an antenna. The device 28 may be an actuator of some sort.

Part or all of the hub 18 may be formed as a printed solid state polymeric battery. That is, the printed solid state polymeric battery may replace part or all of the hub 18. Rather than being formed of the polymers that are traditionally used in forming a catheter hub such as the hub 18, the hub 18 may instead be formed of electroactive polymers (as will be discussed). In some cases, part or all of the strain relief 26 may be formed as a printed solid state polymeric battery. In some cases, conductive traces between a printed solid state polymeric battery forming part or all of the hub 18, the strain relief 26 or both, may be formed on an outer surface of the catheter shaft 12. FIG. 1A shows a printed conductive trace 28 and a printed conductive trace 30 that have been printed on the outer surface of the catheter shaft 12. It will be appreciated that the relative position of the printed conductive traces 28, 30 shown in FIG. 1A is merely illustrative. The printed conductive traces 28, 30 may be printed anywhere on the circumference of the catheter shaft 12, as long as the printed conductive traces 28, 30 are electrically isolated from each other. While not illustrated, the catheter shaft 12 may include an outer layer that protects the printed conductive traces 28, 30 as well as providing desired characteristics such as lubricity and biocompatibility to the catheter shaft 12. As an example, PET (polyethylene terephthalate) shrink tubing such as that available commercially from Nordson Medical of Galway, Ireland may be used.

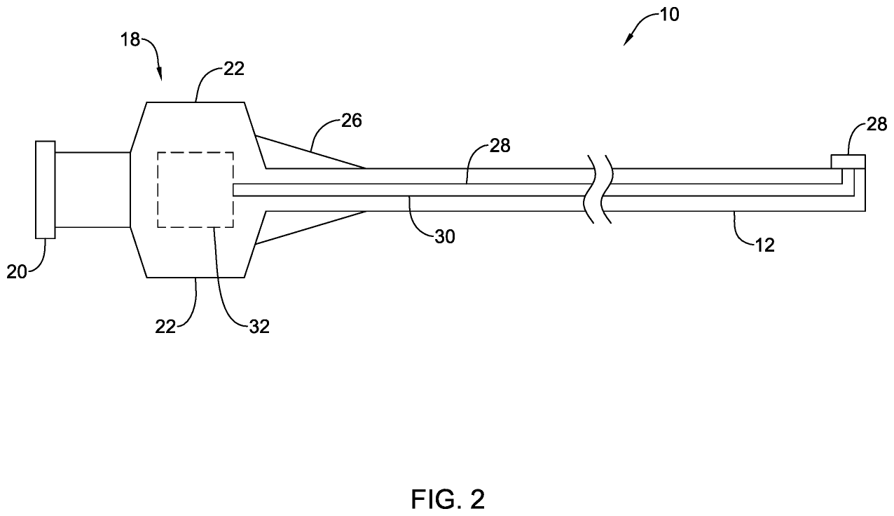

FIG. 2 is a schematic view of the catheter 10 in which a printed solid state polymeric battery 32 is schematically shown as being within the hub 18. In some cases, the printed solid state polymeric battery 32 forms part or all of the hub 18. As seen in FIG. 2, the printed conductive trace 28 and the printed conductive trace 30 can be seen as extending from the printed solid state polymeric battery 32 to the device 30. As a result, the printed solid state polymeric battery 32 is able to provide power for operating the device 30. In some cases, the device 30 may itself control when and how the device 30 receives electrical power from the printed solid state polymeric battery 32. In some instances, the catheter 10 may include a controller (not shown) that is electrically coupled between the device 30 and the printed solid state polymeric battery 32.

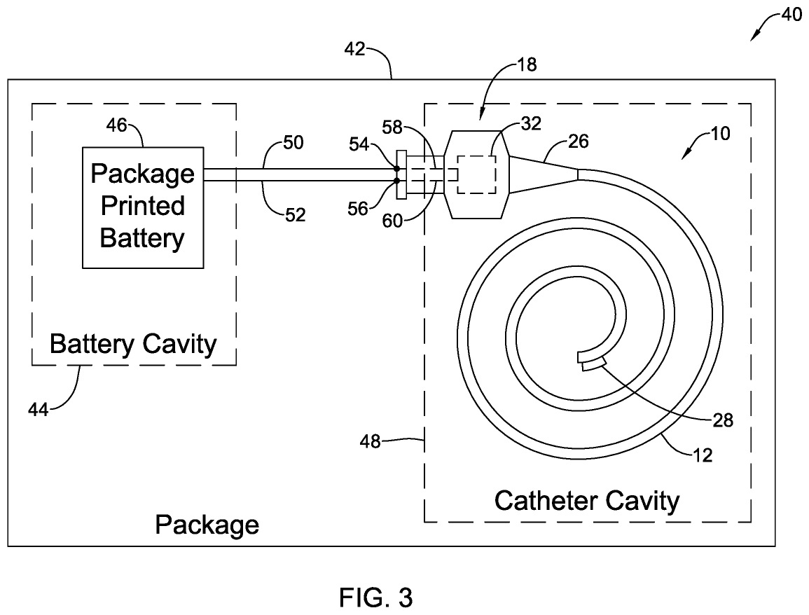

FIG. 3 is a schematic view of a catheter system 40. The catheter system 40 includes the catheter 10 as well as a package 42. The package 42 may be formed of any appropriate material such as a polymeric or even a paper material. The package 42 may be intended for single use, and to be disposed of once the catheter 10 is initially removed from the package 42. In some cases, the package 42 may be a clamshell design, or otherwise have a front portion and a back portion that can be easily separated in order to open the package 42. The package 42 may be adapted, for example, to permit the catheter 10 to be sterilized using any of a number of different sterilization procedures while the catheter 10 is present within the package 42. For example, the catheter 10 may be subjected to a radiative sterilization process such as e beam radiation or gamma radiation while in the package 42. The catheter 10 may be subjected to sterilization via an ethylene oxide atmosphere, for example.

In some cases, the package 42 includes a battery cavity 44 that is adapted to accommodate a printed package battery 46. The package 42 may include a catheter cavity 48 that accommodates the catheter 10. It will be appreciated that the catheter 10 is shown schematically, and in some cases may be coiled up within the catheter cavity 48, as shown. In some cases, depending on the relative dimensions of the catheter 10, the catheter 10 may instead have a linear configuration within the catheter cavity.

The catheter system 40 includes conductive elements 50 and 52 that electrically couple the printed package battery 46 with the printed solid state polymeric battery 32 within the catheter 10. The conductive elements 50 and 52 may, for example, be printed within the package 42. In some instances, the conductive elements 50 and 52 may be formed in any suitable manner, and may for example represent wires disposed within the package 42.

In some cases, for example, power within the printed package battery 46 may be used to charge the printed solid state polymeric battery 32 before use of the catheter 10. In some instances, power from the printed package battery 46 may be used to keep the printed solid state polymeric battery 32 fully charged within the package 40, so that the catheter 10 will be immediately ready for use once the catheter 10 is removed from the package 40. The conductive elements 50 and 52 are adapted to be coupled to conductive elements 54 and 56, which are electrically coupled to the printed solid state polymeric battery 32 via conductors 58 and 60, respectively, which extend through the Luer fitting 20. Another option is to attach two clamp electrode contacts on the package 40. The clamp electrode contacts may be clamped onto the catheter tubing, for example. The anode and cathode may be connected to these contact points via conductive traces.

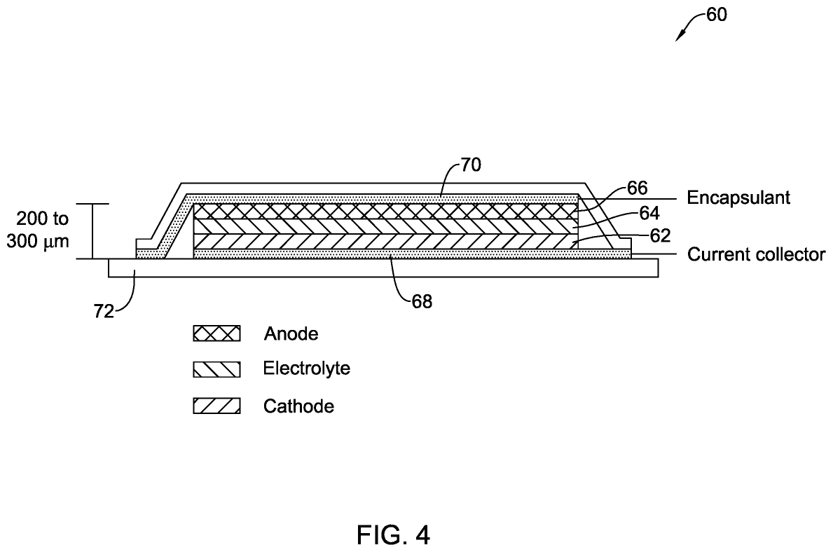

FIG. 4 is a schematic view of a printed solid state polymeric battery 60 such as the printed solid state polymeric battery 32 shown in FIGS. 2 and 3. In some cases, the printed solid state polymeric battery 60 may also be used as the printed package battery 46 (FIG. 3). It will be understood that in some instances, the printed package battery 46 may be physically larger, with a higher electrical capacity, than the printed solid state polymeric battery 32. The printed package battery 46 may also have a larger, flatter profile than the printed solid state polymeric battery 32.

The printed solid state polymeric battery 60 may be formed using any desired printing techniques, such as but not limited to screen printing and 3D printing. In some cases, printed solid state polymeric battery 60 may be printed using appropriate chemical species to render the printed solid state polymeric battery 60 initially uncharged. In some cases, the printed solid state polymeric battery 60 may be printed with chemical species that renders the printed solid state polymeric battery 60 with an initial charge that may represent a full charge or a partial charge. In some cases, the printed solid state polymeric battery 60 may be a zinc-based printed battery, such as those available commercially from Imprint Energy of Alameda, Calif. Additional details regarding a suitable printed battery may be found in U.S. Pat. Nos. 9,076,589 and 9,276,292, both of which are incorporated by reference herein, in their entirety.

In some cases, the printed solid state polymeric battery 60 may be formed by printing each of the layers of the printed solid state polymeric battery 60 using chemical species available from Evonik. As can be seen in FIG. 4, the printed solid state polymeric battery 60 includes a number of layers. The printed solid state polymeric battery 60 includes a printed cathode layer 62, a printed electrolyte layer 64 and a printed anode layer 66. The printed cathode layer 62 is formed on a current collector layer 68 and an encapsulant layer 70 covers the printed anode layer 66. The assembly is covered with an insulative polymeric layer 72. It will be appreciated that each of the layers 62, 64, 66 may be flat or curved, depending on whether the printed solid state polymeric battery 60 is forming part or all of the hub 18 (FIG. 1) or the strain relief 26. The encapsulant layer 70 may provide an overall shape to the printed solid state polymeric battery 60, and thus can impart an appropriate shape depending on where the printed solid state polymeric battery 60 is being used within the proximal region 14 of the catheter 10.

In some cases, the printed cathode layer 62 may include a thianthrene-based polymer as the printed cathode layer 62 and a TCAQ tetracyanoanthraquinodimethane (TCAQ)-based polymer as the printed anode layer 66. In an illustrative but non-limiting example, the printed solid state polymeric battery 60 may include poly(2-vinylthianthrene) as the printed cathode layer 62 while the printed anode layer 66 include poly(2-methacrylamide-TCAQ).

An all-organic battery may be positioned circumferentially around a tubular medical device, such as a catheter. Current collectors such as metallic or carbon films may be deposited on the tubing using a low-temperature curing conductive ink such as but not limited to DUPONT™ PE827 or PE 828 ULTRA-LOW TEMPERATURE CURE SILVER COMPOSITE CONDUCTOR, which adheres well to a variety of polymers.

To deposit the ink on a tubular device such as catheter tubing, the catheter may be mounted in a rotating head of a micro-dispensing system from MickroFab Technologies, (1104 Summit Ave., Suite 110 Plano Tex. 75074 U.S.A) to deposit a 10 micrometer thick layer. After curing for 20 minutes at 100 degrees C., the same dispensing head and setup may be used to deposit the anode material ink in a layer 200 micrometers thick on top of the current collector. The anode material ink may be Tattooez, which is available commercially from Evonik, Lipper Weg 190 45772 Marl Haus, Germany. The deposited anode material ink may be dried for 10 minutes at 100 degrees C. Using the same equipment, the electrolyte layer may be deposited on top of the anode layer. The electrolyte layer may be 50 micrometers thick and may be cured using a UV lamp (Ring type UV LED curing lamp Model No.: UVSS-13T: company UVET: 1802, Bldg A, Baian Center, Nancheng District, Dongguan 523073, GuangDong, China) for 2 minutes at a setting of 1.5 Watts. The cathode layer ink 200 micrometer (Evonik) may be deposited on the electrolyte layer and cured for 20 minutes at 100 degrees C., after which a second current collector layer (10 micrometer) may be deposited and cured similar to the first layer.

The precise movement of the tubular device may be accomplished using a combination of two coupled motorized rotational 360 degrees stages (RV120BPP-F Motorized Rotation Stage, mounted on a motorized linear stage MFA-PPD Motorized Linear Stage, Miniature, 25 mm Travel, Stepper Motor (all from Newport Spectra-Physics BV, Vechtensteinlaan 12-16, 3555 XS Utrecht, Netherlands). The catheter tubing may be held at both rotational heads by a chuck. Using this setup (translation and rotation) allows virtual any 3D shape to be printed. For example by printing more material (anode+cathode) on opposite sides of the tubular structure allows to create a tube with wings allowing for a better grip.

Other polymeric materials that may be used for the cathode or anode include polyacetylene and polypyrroles (PPY). In some cases, PPY may be utilized as a cathode material (i.e., oxidized during charging). However, PPY can also be reduced, allowing for its use as an anode material. Polymer-based batteries with two PPY electrodes are therefore pol-less, meaning the electrodes can be utilized as both anode and cathode. Polythiophene (PT), poly(ethylenedioxythiophene) (PEDOT), and other alkoxy-substituted polythiophenes can be used as polymeric anodes for example in combination with PPY as cathode. Another class of materials are carbonyl compounds, in particular quinones and imides. For example hydroquinones as well as anthraquinones can be used as an anode.

Materials that may be used as a printable solid state electrolyte include an ionic liquid (for example: EMImTFSI, BMImTFSI, EMIm triflate, and BMIm triflate) gelled in a methacrylate-based polymer matrix, which can be printed and crosslinked by UV on existing polymeric electrodes as described by Simon Muench et al.; Printable ionic liquid-based gel polymer electrolytes for solid state; Energy Storage Materials.

Additional non-electrical conductive materials can be added as functional reinforcement materials Examples include nano- or micro particles such as zirconia, glass, silica, polystyrene. Suitable materials are available from Microspheres; 3590 Route 9, Suite 107 Cold Spring, N.Y. 10516. https://www.microspheres-nanospheres.com/Microspheres/Inorganic/Glass/Glass.htm).

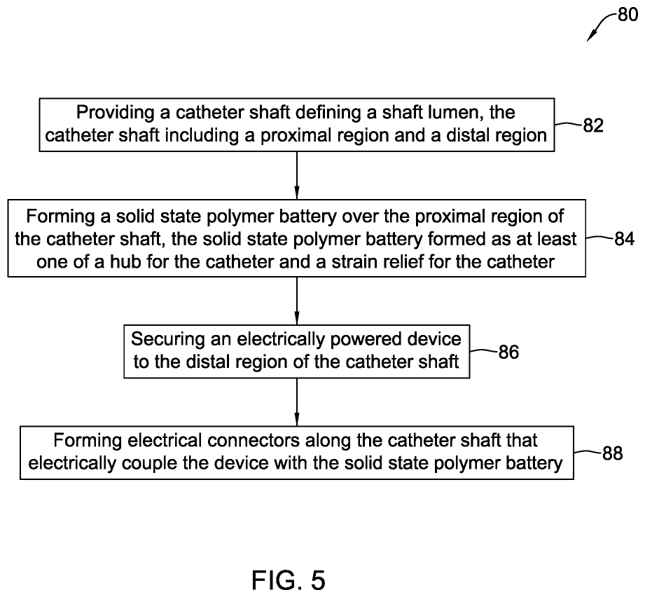

FIG. 5 is a flow diagram showing an illustrative method 80 of manufacturing the catheter 10. A catheter shaft such as the catheter shaft 12 is provided that defines a shaft lumen such as the shaft lumen 24 (FIG. 1A), as indicated at block 82. A solid state polymer battery is formed over the proximal region of the catheter shaft, the solid state polymer battery formed as at least one of a hub for the catheter and a strain relief for the catheter, as indicated at block 84. An electrically powered device is secured to the distal region of the catheter shaft, as indicated at block 86. Electrical connectors are formed along the catheter shaft that electrically couple the device with the solid state polymer battery, as indicated at block 88.

In some cases, forming the solid state polymer battery comprises providing or printing a current collector layer. A cathode layer is printed on the current collector layer. An electrolyte layer is printed on the cathode layer. An anode layer is printed on the electrolyte layer. A current collector layer is printed on the anode layer. In some cases, one or more encapsulating layers may be formed over the printed layers. The one or more encapsulating layer may form at least a portion of an outer profile of the at least the hub or the strain relief.

It will be appreciated that a variety of different materials may be used in forming the packaging described herein. In some embodiments, for example, the packaging materials may include any suitable polymeric material, including biocompatible materials such as polyurethane or silicone. Other suitable polymers include but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A catheter, comprising:
a shaft extending from a proximal region to a distal region and defining a shaft lumen extending therethrough;
a hub secured relative to the proximal region; and
a strain relief extending proximally over the shaft from the hub;
wherein at least one of the hub and the strain relief comprises a printed battery.

2. The catheter of claim 1, wherein the hub comprises the printed battery.

3. The catheter of claim 2, wherein the hub includes hub wings forming a portion of the printed battery.

4. The catheter of claim 2, wherein the hub includes a Luer fitting that provides an electrical connection to the printed battery.

5. The catheter of claim 4, wherein the Luer fitting also provides a fluid coupling to the shaft lumen.

6. The catheter of claim 1, further comprising a device coupled to the catheter that is powered by the printed battery.

7. The catheter of claim 6, wherein the device comprises one or more of a sensor, an actuator and an antenna.

8. The catheter of claim 6, further comprising printed conductive traces that extend from the printed battery to the device in order to power the device.

9. The catheter of claim 1, wherein the strain relief comprises the printed battery.

10. The catheter of claim 1, wherein the printed battery comprises:
a printed current collector layer;
a printed cathode layer;
a printed electrolyte layer; and
a printed anode layer.

11. The catheter of claim 10, wherein the printed battery further comprises an encapsulating layer that forms at least a portion of an outer profile of the at least the hub or the strain relief.

12. A catheter system, comprising:
a catheter including:
a shaft extending from a proximal region to a distal region and defining a shaft lumen extending therethrough;
a hub secured relative to the proximal region; and
a strain relief extending proximally over the shaft from the hub;
wherein at the hub comprises a printed catheter battery; and
a package adapted to secure the catheter within the package, the package including:
a cavity adapted to accommodate the catheter; and
a printed package battery disposed within the package, the printed package battery electrically coupled with the printed catheter package in order to maintain an electrical charge within the printed catheter battery.

13. The catheter system of claim 12, wherein the catheter includes a Luer fitting that is electrically coupled with the printed catheter battery, and the printed package battery is electrically coupled with the printed catheter battery via the Luer fitting.

14. The catheter system of claim 13, wherein the Luer fitting also provides a fluid coupling with the shaft lumen.

15. The catheter system of claim 12, wherein the catheter further comprises a device coupled to the catheter that is powered by the printed catheter battery.

16. The catheter system of claim 15, wherein the device comprises one or more of a sensor, an actuator and an antenna.

17. The catheter system of claim 15, wherein the catheter further comprises printed conductive traces that extend from the printed catheter battery to the device in order to power the device.

18. A method of manufacturing a catheter, the method comprising:
providing a catheter shaft defining a shaft lumen, the catheter shaft including a proximal region and a distal region;
forming a solid state polymer battery over the proximal region of the catheter shaft, the solid state polymer battery formed as at least one of a hub for the catheter and a strain relief for the catheter;
securing an electrically powered device to the distal region of the catheter shaft; and
forming electrical connectors along the catheter shaft that electrically couple the device with the solid state polymer battery.

19. The method of claim 18, wherein forming the solid state polymer battery comprises:
printing a current collector layer;
printing a cathode layer;
printing an electrolyte layer; and
printing an anode layer.

20. The method of claim 19, further comprising forming an encapsulating layer over the printed layers that forms at least a portion of an outer profile of the at least the hub or the strain relief.
